(19) United States
(12) Patent Application Publication
(10) Pub. No.: US 2003/0167516 A1
(43) Pub. Date: Sep. 4, 2003

(54) Calcium dependent protein kinase polypeptides as regulators of plant disease resistance

(76) Inventor: Jen Sheen, Boston, MA (US)

Correspondence Address: CLARK & ELBING LLP, 101 FEDERAL STREET, BOSTON, MA, 02110, US

(21) Appl. No.: 09/848,806

(22) Filed: May 4, 2001

Related U.S. Application Data
(60) Provisional application No. 60/201,925, filed on May 5, 2000.

(51) Int. Cl. A01H 5/00

(52) U.S. Cl. 800/279

(57) ABSTRACT

In general, the invention features a method of producing a plant having increased disease resistance. The method includes the steps of: (a) providing a non-naturally occurring plant cell overexpressing a nucleic acid molecule encoding a calcium dependent protein kinase (CDPK) polypeptide; and (b) regenerating a plant from said plant cell, wherein the CDPK polypeptide is expressed in said plant, increasing the resistance of the plant to disease as compared to a naturally-occurring plant. In addition, the invention In related aspects, the invention further features non-naturally occurring plants (or plant cell, plant tissue, plant organ, or plant component) that expresses a nucleic acid molecule encoding either a CDPK2 or CDPK4 polypeptides or both, as well as seeds and cells of such plants.

This application claims benefit of U.S. provisional patent application 60/201,925, filed on May 5, 2000, which is hereby incorporated by reference in its entirety.

### BACKGROUND OF THE INVENTION

The invention relates to plant disease resistance.

Many agriculturally important crops are susceptible to disease caused by a variety of pathogens including viruses, bacteria, fungi, and nematodes. Such pathogens seriously damage a crop and reduce its economic value to the grower. Crop plants are particularly vulnerable, and when disease strikes, losses can be severe. Attempts to control or prevent infection and disease of crop plants have been made, but until recently none have been completely satisfactory. Thus, although natural variation for resistance to plant pathogens has been identified by plant breeders and pathologists and bred into many crop plants, the development of disease resistant plants, particulary plants exhibiting broad spectrum disease resistance remains an import goal in agriculture. A need therefore exists for new methods of protecting plants from their pathogens

### SUMMARY OF THE INVENTION

The invention is based on applicant's discovery that a calcium dependent protein kinase (CDPK) polypeptide, a CDPK2 polypeptide of Arabidopsis, is involved in signaling the activation of early pathogen response genes. Such CDPKs, therefore, serve as master regulators of pathogen responsive genes, serving to enhance multiple pathogen resistance. Accordingly, the invention involves methods of genetically engineering disease resistant plants.

In one aspect, the invention therefore features a method of producing a plant having increased disease resistance. The method, in general, includes the steps of: (a) providing a non-naturally occurring plant cell overexpressing a nucleic acid molecule encoding a calcium dependent protein kinase (CDPK) polypeptide; and (b) regenerating a plant from the plant cell, wherein the CDPK polypeptide is expressed in the plant, increasing the resistance of the plant to disease as compared to a naturally-occurring plant. In preferred embodiments, the plant cell is a dicotyledonous plant cell (e.g., a cruciferous plant cell) or a monocotyledonous plant cell. In particular applications, the method is especially useful for providing resistance to a disease that is caused by a plant pathogen. In other preferred embodiments, the non-naturally occurring plant cell is a transgenic plant cell. Preferably, such a plant cell includes a transgene that expresses a nucleic acid molecule encoding a CDPK polypeptide (for example, CDPK2 or CDPK4 polypeptides or both). In still other preferred embodiments, the transgenic plant cell includes a CDPK polypeptide that consists essentially of the CDPK protein kinase domain. In preferred embodiments, the transgene ectopically expresses the nucleic acid molecule encoding the CDPK polypeptide. Preferably, the transgene includes either an inducible, constitutive, or tissue-specific promoter. In still other preferred embodiments, the nucleic acid molecule is either derived from Arabidopsis or is an ortholog thereof.

In another aspect, the invention features a method of conferring pathogen resistance on a plant. The method, in general, includes the steps of (a) crossing a pathogen resistant plant prepared by the above-described method with a plant having susceptibility to a pathogen; (b) recovering reproductive material from the progeny of the cross; and (c) growing pathogen resistant plants from the reproductive material. In preferred embodiments, the method further includes repetitively crossing the pathogen resistant progeny with disease susceptible plants, and selecting for expression of pathogen resistance.

In another aspect, the invention features a method for breeding pathogen resistance into plants. The method, in general, includes the steps of (a) selecting a plant that expresses a nucleic acid molecule encoding a CDPK polypeptide; and (b) selecting pathogen resistant progeny. In preferred embodiments, the plant is a transgenic plant. Such transgenic plants include transgenes that express nucleic acid molecules encoding CDPK polypeptides. In preferred embodiments, the transgene ectopically expresses a nucleic acid molecule encoding the CDPK polypeptide (for example, CDPK2 or CDPK4 polypeptides or both). In yet other preferred embodiments, the CDPK polypeptide consists essentially of the CDPK protein kinase domain.

In still another aspect, the invention features a non-naturally occurring plant (or plant cell, plant tissue, plant organ, or plant component) that expresses a nucleic acid molecule encoding either a CDPK2 or CDPK4 polypeptide. In preferred embodiments, the plant includes both CDPK2 and CDPK4 polypeptides. In other preferred embodiments, the plant includes a transgene that includes a nucleic acid molecule encoding a CDPK2 or CDPK4 polypeptide, expression of the nucleic acid molecule being under the control of an expression control region that is functional in a plant cell. Preferably, the nucleic acid molecule encoding the CDPK2 or CDPK4 polypeptide is derived from a plant. In still other preferred embodiments, the CDPK2 or CDPK4 polypeptides consist essentially of the protein kinase domain, and the transgene encoding each of these polypeptides is either derived from Arabidopsis or is an ortholog thereof. In other preferred embodiments, the plant is a dicotyledonous or monocotyledonous plant.

In related aspects, the invention features seeds and cells from the non-naturally occurring plant that express a CDPK2 polypeptide or a CDPK4 polypeptide or both.

In still other related aspects, the invention features a vector (for example, an expression vector) including an expression control region functional in plant cells operably linked to a nucleic acid molecule encoding a CDPK2 polypeptide or a CDPK4 polypeptide. In preferred embodiments, the CDPK2 or CDPK4 polypeptides consist essentially of the protein kinase domain. In other preferred embodiments, the nucleic acid molecule encoding the CDPK2 or CDPK4 polypeptides is derived from a plant. In other preferred embodiments, the nucleic acid molecule that encodes the CDPK2 or CDPK4 polypeptide is either derived from Arabidopsis or is an ortholog thereof. In yet other preferred embodiments, the vector includes a nucleic acid molecule that expresses both a CDPK2 and CDPK4 polypeptide, or a CDPK protein kinase domain.

In related aspects, the invention also includes a cell that includes any of the above-mentioned vectors.

In general, the protein kinase domain used in the methods or plants (e.g., transgenic plants or plants that are bred using a transgenic plant) of the invention is generally expressed by itself, as a CDPK polypeptide or protein kinase domain-containing fragment thereof, or as part of a genetically engineered chimeric polypeptide. Useful constitutively-active protein kinase domains include those that are capable of activating a gene involved in a pathogen defense response. Exemplary protein kinase domains include, without limitation, those that are substantially identical to the protein kinase domains of CDPK2 or CDPK4. Preferably, the methods and plants of the invention specifically utilize the protein kinase domain of CDPK2 or CDPK4 or both. In other preferred embodiments, a full-length CDPK polypeptide or a protein kinase domain-containing fragment thereof that is substantially identical to any one of CDPK2 or CDPK4 is utilized.

The DNA encoding the protein kinase domain is, in general, constitutively expressed. However, if desired, the protein kinase domain is inducibly expressed, or such a domain is expressed in a cell-specific, tissue-specific, or organ-specific manner. Moreover, the kinase domain can also be expressed under cycling conditions (e.g., cell cycle or circadian conditions).

By “nucleic acid molecule” is meant a DNA or RNA molecule or sequence, and may include, for example, a cDNA, genomic DNA, or synthetic DNA sequence, a structural gene or a fragment thereof, or an mRNA sequence, that encodes an active or functional polypeptide.

By “polypeptide” is meant any chain of amino acids, regardless of length or post-translational modification (for example, glycosylation or phosphorylation).

By “substantially identical” is meant a polypeptide or nucleic acid exhibiting at least 40%, preferably 50%, more preferably 80%, and most preferably 90%, or even 95% sequence identity to a reference sequence (for example, the amino acid sequences of the kinase domains or full-length CDPK polypeptides of CDPK2 or CDPK4 or to their respective nucleic acid sequences (FIGS. 1 and 2). For polypeptides, the length of comparison sequences will generally be at least 16 amino acids, preferably at least 20 amino acids, more preferably at least 25 amino acids, and most preferably 35 amino acids or greater. For nucleic acids, the length of comparison sequences will generally be at least 50 nucleotides, preferably at least 60 nucleotides, more preferably at least 75 nucleotides, and most preferably 110 nucleotides or greater.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, FASTA, PILEUP/PRETTYBOX programs, or other publicly available sequence analysis programs, for example those found at http://www.expasy.ch/tools). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “derived from” is meant isolated from or having the sequence of a naturally-occurring sequence (for example, a cDNA, genomic DNA, synthetic DNA, or combination thereof).

By “transformed cell” is meant a cell into which (or into an ancestor of which) has been introduced, by means of recombinant DNA techniques, a DNA molecule encoding (as used herein) a calcium dependent protein kinase polypeptide.

By “reporter gene” is meant a gene whose expression may be assayed; such genes include, without limitation, β-glucuronidase (GUS), luciferase, chloramphenicol transacetylase (CAT), green fluorescent protein (GFP), β-galactosidase, herbicide resistant genes, and antibiotic resistance genes.

By “expression control region” is meant any minimal sequence sufficient to direct transcription. Included in the invention are promoter elements that are sufficient to render promoter-dependent gene expression controllable for cell-, tissue-, or organ-specific gene expression, or elements that are inducible by external signals or agents (for example, light-, pathogen-, wound-, stress-, or hormone-inducible elements or chemical inducers such as SA or INA); such elements may be located in the 5′ or 3′ regions of the native gene or engineered into a transgene construct.

By “operably linked” is meant that a gene and a regulatory sequence(s) are connected in such a way as to permit gene expression when the appropriate molecules (for example, transcriptional activator proteins) are bound to the regulatory sequence(s).

By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores.

By “plant component” is meant a part, segment, or organ obtained from an intact plant or plant cell. Exemplary plant components include, without limitation, explant, somatic embryos, leaves, fruits, scions, and rootstocks.

By “crucifer” is meant any plant that is classified within the Cruciferae family. The Cruciferae include many agricultural crops, including, without limitation, rape (for example, Brassica campestris and Brassica napus), broccoli, cabbage, brussel sprouts, radish, kale, Chinese kale, kohlrabi, cauliflower, turnip, rutabaga, mustard, horseradish, and Arabidopsis.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell. As used herein, the transgenic organisms are generally transgenic plants and the DNA (transgene) is inserted by artifice into the nuclear or plastidic genome. A transgenic plant according to the invention may contain one or more calcium dependent protein kinase genes.

By “ectopically expressed” is meant a nucleic acid molecule encoding a polypeptide, for example, polypeptide that includes a constitutively-active CDPK protein kinase domain, that is expressed in a tissue other than a tissue in which it normally is expressed or at a time other than the time at which it normally is expressed. Ectopic expression of the polypetide may, for example, also be constitutive or inducible.

By “pathogen” is meant an organism whose infection of viable plant tissue elicits a disease response in the plant tissue. Such pathogens include, without limitation, bacteria, mycoplasmas, fungi, oomycetes, insects, nematodes, viruses, and viroids. Examples of such plant diseases are described in Chapters 11-16 of Agrios, Plant Pathology, 3rd ed., Academic Press, Inc., New York, 1988.

Examples of bacterial pathogens include, without limitation, Erwinia (for example, E. carotovora), Pseudomonas (for example, P. syringae), and Xanthomonas (for example, X. campepestris and X. oryzae).

Examples of fungal or fungal-like disease-causing pathogens include, without limitation, Alternaria (for example, A. brassicola and A.solani), Ascochyta (for example, A. pisi), Botrytis (for example, B. cinerea), Cercospora (for example, C. kikuchii and C. zaea-maydis), Colletotrichum sp. (for example, C. lindemuthianum), Diplodia (for example, D. maydis), Erysiphe (for example, E. graminis f.sp. graminis and E. graminis f.sp. hordei), Fusarium (for example, F. nivale and F. oxysporum, F. graminearum, F. solani, F. monilforme, and F. roseum), Gaeumanomyces (for example, G. graminis f.sp. tritici), Helminthosporium (for example, H. turcicum, H. carbonum, and H. maydis), Macrophomina (for example, M. phaseolina and Maganaporthe grisea), Nectria (for example, N. heamatocacca), Peronospora (for example, P. manshurica, P. tabacina), Phoma (for example, P. betae), Phymatotrichum (for example, P. omnivorum), Phytophthora (for example, P. cinnamomi, P. cactorum, P. phaseoli, P. parasitica, P. citrophthora, P. megasperma f.sp. sojae, and P. infestans), Plasmopara (for example, P. viticola), Podosphaera (for example, P. leucotricha), Puccinia (for example, P. sorghi, P. striiformis, P. graminis f.sp. tritici, P. asparagi, P. recondita, and P. arachidis), Puthium (for example, P. aphanidermatum), Pyrenophora (for example, P. tritici-repentens), Pyricularia (for example, P. oryzea), Pythium (for example, P. ultimum), Rhizoctonia (for example, R. solani and R. cerealis), Scerotium (for example, S. rolfsii), Sclerotinia (for example, S. sclerotiorum), Septoria (for example, S. lycopersici, S. glycines, S. nodorum and S. tritici), Thielaviopsis (for example, T. basicola), Uncinula (for example, U. necator), Venturia (for example, V. inaequalis), Verticillium (for example, V. dahliae and V. albo-atrum).

Examples of pathogenic nematodes include, without limitation, root-knot nematodes (for example, Meloidogyne sp. such as M. incognita, M. arenaria, M. chitwoodi, M. hapla, M. javanica, M. graminocola, M. microtyla, M. graminis, and M. naasi), cyst nematodes (for example, Heterodera sp. such as H. schachtii, H. glycines, H. sacchari, H. oryzae, H. avenae, H. cajani, H. elachista, H. goettingiana, H. graminis, H. mediterranea, H. mothi, H. sorghi, and H. zeae, or, for example, Globodera sp. such as G. rostochiensis and G. pallida), root-attacking nematodes (for example, Rotylenchulus reniformis, Tylenchuylus semipenetrans, Pratylenchus brachyurus, Radopholus citrophilus, Radopholus similis, Xiphinema americanum, Xiphinema rivesi, Paratrichodorus minor, Heterorhabditis heliothidis, and Bursaphelenchus xylophilus), and above-ground hematodes (for example, Anguina funesta, Anguina tritici, Ditylenchus dipsaci, Ditylenchus myceliphagus, and Aphenlenchoides bessey).

Examples of viral pathogens include, without limitation, tobacco mosaic virus, tobacco necrosis virus, potato leaf roll virus, potato virus X, potato virus Y, tomato spotted wilt virus, and tomato ring spot virus.

By “increased level of resistance” is meant a level of resistance to a disease-causing pathogen in a non-naturally occurring plant (or cell or seed thereof) which is greater than the level of resistance in a control plant (for example, a non-transgenic plant or wild-type). In preferred embodiments, the level of resistance in a non-naturally occurring plant of the invention is at least 20% (and preferably 30% or 40%) greater than the resistance exhibited by a control plant. In other preferred embodiments, the level of resistance to a disease-causing pathogen is 50% greater, 60% greater, and more preferably even 75% or 90% greater than a control plant; with up to 100% or greater above the level of resistance as compared to a control plant being most preferred. The level of resistance is measured using conventional methods. For example, the level of resistance to a pathogen may be determined by comparing physical features and characteristics (for example, plant height and weight, or by comparing disease symptoms, for example, delayed lesion development, reduced lesion size, leaf wilting and curling, water-soaked spots, amount of pathogen growth, and discoloration of cells) of the non-naturally occurring plant (e.g., a transgenic plant).

By “detectably-labeled” is meant any direct or indirect means for marking and identifying the presence of a molecule, for example, an oligonucleotide probe or primer, a gene or fragment thereof, or a cDNA molecule or a fragment thereof. Methods for detectably-labeling a molecule are well known in the art and include, without limitation, radioactive labeling (for example, with an isotope such as ³²P or ³⁵S) and nonradioactive labeling (for example, fluorescence of chemiluminescent labeling, for example, fluorescein labeling).

The invention provides a number of important advances and advantages for the protection of plants against their disease-causing pathogens. For example, by expressing CDPK gene products in all species of plants, the invention facilitates an effective and economical means for in-plant protection against plant pathogens. Such protection against pathogens reduces or minimizes the need for traditional chemical practices (for example, application of fungicides, bactericides, nematicides, insecticides, or viricides) that are typically used by farmers for controlling the spread of plant pathogens and providing protection against disease-causing pathogens. In addition, because plants expressing one or more acquired resistance gene(s) described herein are less vulnerable to pathogens and their diseases, the invention further provides for increased production efficiency, as well as for improvements in quality and yield of crop plants and ornamentals. Thus, the invention contributes to the production of high quality and high yield agricultural products: for example, fruits, ornamentals, vegetables, cereals and field crops having reduced spots, blemishes, and blotches that are caused by pathogens; agricultural products with increased shelf-life and reduced handling costs; and high quality and yield crops for agricultural (for example, cereal and field crops), industrial (for example, oilseeds), and commercial (for example, fiber crops) purposes.

Furthermore, because the invention reduces the necessity for chemical protection against plant pathogens, the invention benefits the environment where the crops are grown. Genetically-improved seeds and other plant products that are produced using plants expressing the genes described herein also render farming possible in areas previously unsuitable for agricultural production. The invention further provides a means for mediating the expression of early pathogen responsive genes, for example, phenylalanine ammonia-lyase (PAL) and glutathione S-transferase (GST), that confer resistance to plant pathogens. For example, transgenic plants constitutively producing a constitutively-active CDPK gene product are capable of activating gene expression, which, in turn, confers resistance to plant pathogens. Collective activation of early pathogen response gene expression that is mediated by the CDPK gene product obviates the need to express these genes individually as a means to promote plant defense mechanisms.

Other features and advantages of the invention will be apparent from the following description of the preferred embodiments thereof, and from the claims.

### DETAILED DESCRIPTION

The drawings will first be described.

### DRAWINGS

FIG. 1 shows the amino acid sequence (SEQ ID NO: 1) and corresponding nucleotide sequence (SEQ ID NO: 2) of CDPK2 of Arabidopsis thaliana. The “atg” start codon is underlined.

FIG. 2 shows the amino acid sequence (SEQ ID NO: 3) and corresponding nucleotide sequence (SEQ ID NO: 4) of CDPK4 of Arabidopsis thaliana. The “atg” start codon is underlined.

One important plant early response to diverse pathogens is the activation of early responsive genes that are essential for plant defense to compatible and incompatible pathogens. It is known that one of the early plant responses to pathogens is the elevation of calcium signals. It is, however, not clear what the calcium sensors are and what the downstream signaling cascade is, and how important it is for plant defense. Using an Arabidopsis protoplast transient expression system, I have found that a specific calcium-dependent protein kinase, CDPK2, and its functionally redundant homolog, CDPK4, activate several well-defined early pathogen responsive genes. Indeed, the Arabidopsis CDPK2 is the first plant gene that has been shown in vivo to be directly involved in the activation of early pathogen responsive genes in a functional assay.

In particular, I have used an Arabidopsis protoplast transient expression system and four reporter genes to identify CDPKs that activate early pathogen responsive genes. The constitutively-active CDPK2 activate CHS, PAL1, GST1, and GCC1. Thus, CDPK2 is a useful target for manipulation to generate agronomically valuable traits, such as multiple pathogen resistance. The expression of the CHS gene regulates a key enzyme in the biosynthesis of diverse flavonoids involved in disease resistance. The expression of the PAL1 gene is a universal key step for the biosynthesis of salicylic acid and chemolexin, anti-pathogen hormone and chemical. The expression of GST1 is important for the protection and detoxification of cells during pathogen infection. And the activation of the GCC1 enhancer is important for ethylene signaling, another essential hormone for pathogen responses.

Accordingly, evidence is presented below showing that a constitutively active calcium dependent protein kinase (CDPK) polypeptide, CDPK2, activates early pathogen response genes bypassing pathogen signals. The results indicate that CDPK (including their protein kinase domains) play distinct physiological roles. CDPK2 is therefore an example of a positive regulator, useful for controlling pathogen signal transduction in plants. Expression of such regulators in plants is useful for turning on the early pathogen response signal transduction pathway as a means for increasing disease resistance.

The examples provided below are for the purpose of illustrating the invention, and should not be construed as limiting.

Constitutively-Active CDPK2 Activates Pathogen Early Response-Inducible Promoters

To determine whether CDPK2 (Urao et al., Mol. Gen. Genet. 244: 331-340, 1994) is involved in disease resistance, I tested the effect of a constitutively active CDPK2 polypeptide on the activity of several inducible promoters activated in early pathogen defense. The promoters tested were the chalcone synthase (“CHS”) promoter (Feinbaum et al., Mol. Cell Biol. 8: 1985-1992, 1988); the phenylalanine lyase (“PAL1”) promoter (Ohl et al., Plant Cell 2: 837-848, 1990); the ethylene enhancer element (“GCC1”) (Fujimoto et al., Plant Cell 12: 393-404, 2000); and the glutathione S-transferase promoter (“GST1”) (Yang et al., Plant Cell Rep. 17: 700-704, 1999). Chimeric genes were generated by fusing the inducible promoters to a firefly luciferase sequence (Sheen, Science 274: 1900-1902, 1996; Kotvun et al., Proc. Natl. Acad. Sci. 97: 2940-2945, 2000). The CDPK2 polypeptide including the protein kinase domain was cloned into a plant expression vector with a derivative of the 35S promoter and the nos terminator (Sheen, Science 274: 1900-1902, 1996) using standard methods. Arabidopsis protoplasts were co-transfected with one of the dicot promoter firefly luciferase reporter gene constructs and the CDPK2 construct, and assayed according to the standard methods (Sh, Science 274: 1900-1902, 1996; Kotvun et al., Proc. Natl. Acad. Sci. 97: 2940-5,2000). The constitutively active CDPK2 polypeptide was found to activate the expression of the CHS, PAL1, GCC1, and GST promoters. Constitutive expression of a mutated CDPK2 polypeptide, APK1, or the serine-threonine protein kinase, ASK1 (Sheen, Science 274: 1900-1902, 1996) had no effect on the expression of the dicot reporter genes. Similar results were found when constitutively-active CDPK4 was used.

Isolation of Sequences Encoding CDPK Polypeptide Regulators of Disease Resistance

The isolation of additional CDPK polypeptides, as well as CDPK protein kinase domains, having the ability to regulate early pathogen response genes in plants is accomplished using standard strategies and techniques that are well known in the art.

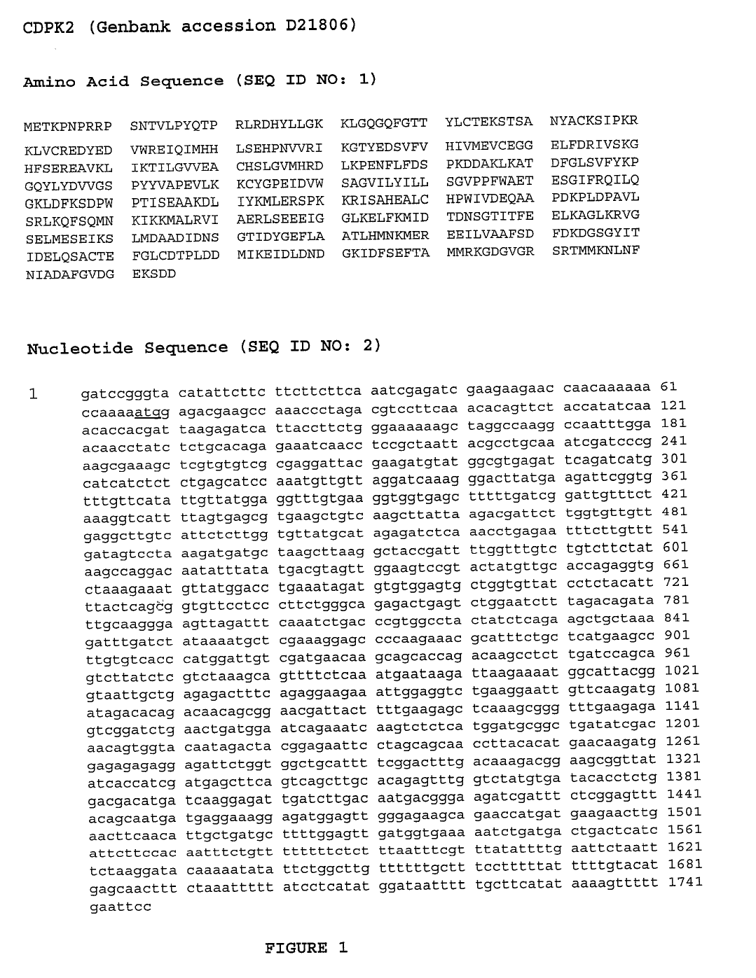

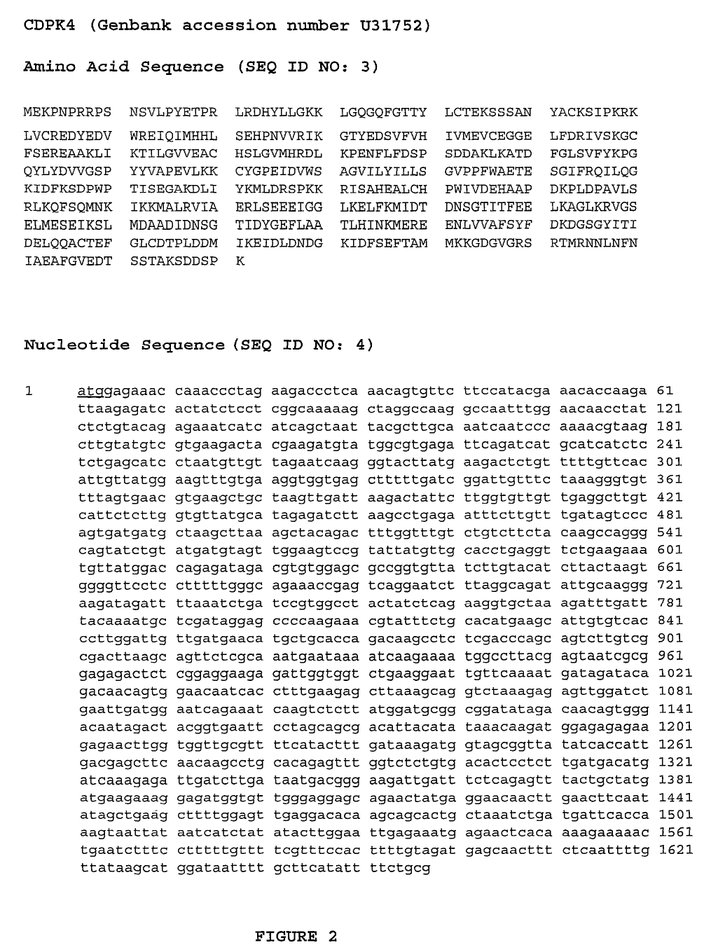

In one particular example, the Arabidopsis CDPK2 sequences (FIG. 1) (or homologs or orthologs or functional equivalents (such as CDPK4 thereof (FIG. 2)) described herein may be used, together with conventional screening methods of nucleic acid hybridization screening, to isolate additional sequences encoding CDPK polypeptides (or protein kinase domain-containing fragments thereof), as well as protein kinase domains of CDPK polypeptides. Such hybridization techniques and screening procedures are well known to those skilled in the art and are described, for example, in Benton and Davis, Science 196: 180, 1977; Grunstein and Hogness, Proc. Natl. Acad. Sci., USA 72: 3961, 1975; Ausubel et al. Current Protocols in Molecular Biology, Wiley Interscience, New York; Berger and Kimmel, Guide to Molecular Cloning Techniques, 1987, Academic Press, New York.; and Sambrook et al., Molecular Cloning: A Laboratory Manual, Cold Spring Harbor Laboratory Press, New York. In one particular example, all or part of the CDPK2 gene (described herein) may be used as a probe to screen a recombinant plant DNA library for genes having sequence identity or similarity to the CDPK gene or its protein kinase domain. Hybridizing sequences are detected by plaque or colony hybridization according to the methods described below.

Alternatively, using all or a portion of the amino acid sequence of the protein kinase domain, one may readily design protein kinase domain-specific oligonucleotide probes, including protein kinase domain degenerate oligonucleotide probes (i.e., a mixture of all possible coding sequences for a given amino acid sequence). These oligonucleotides may be based upon the sequence of either DNA strand and any appropriate portion of the kinase domain sequence. General methods for designing and preparing such probes are provided, for example, in Ausubel et al., Current Protocols in Molecular Biology, Wiley Interscience, New York; and Berger and Kimmel, Guide to Molecular Cloning Techniques, 1987, Academic Press, New York. These oligonucleotides are useful for protein kinase domain sequence isolation, either through their use as probes capable of hybridizing to protein kinase complementary sequences or as primers for various amplification techniques, for example, polymerase chain reaction (PCR) cloning strategies. If desired, a combination of different oligonucleotide probes may be used for the screening of a recombinant DNA library. The oligonucleotides may be detectably-labeled using methods known in the art and used to probe filter replicas from a recombinant DNA library. Recombinant DNA libraries are prepared according to methods well known in the art, for example, as described in Ausubel et al. (supra), or they may be obtained from commercial sources.

As discussed above, protein kinase domain-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, protein kinase domain sequences may be isolated using the PCR “RACE” technique, or Rapid Amplification of cDNA Ends (see, e.g., Innis et al. (supra)). By this method, oligonucleotide primers based on a protein kinase domain sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci. USA 85: 8998, 1988.

Confirmation of a sequence's relatedness to the protein kinase domains of CDPK2 (or homologs or orthologs or functional equivalents (such as CDPK4) thereof) may be accomplished by a variety of conventional methods including, but not limited to, sequence comparison of the gene and its expressed product. In addition, the activity of the gene product may be evaluated according to any of the techniques described herein, for example, by analyzing the expression of a reporter gene under the control of an early pathogen defense gene in the presence of a constitutively-active CDPK polypeptide using either the maize (Sheen, Science 274: 1900-1902, 1996) or Arabidopsis transient protoplast assay systems (Kotvun et al., Proc. Natl. Acad. Sci. 97: 2940-29405, 2000).

In addition, the CDPK2 and CDPK4 sequences disclosed herein provide a basis for searching databases such as Genbank to identify CDPK homologs and orthologs and functional equivalents.

Once a CDPK gene or its protein kinase domain is identified, it is cloned according to standard methods and used for the construction of plant expression vectors as described below.

Expression Constructs

Those skilled in the field of molecular biology will understand that any of a wide variety of expression systems may be used to provide the recombinant protein. The precise host cell used is not critical to the invention. A CDPK polypeptide or its protein kinase domain may be produced in a prokaryotic host, for example, E. coli, or in a eukaryotic host, for example, Saccharomyces cerevisiae, mammalian cells (for example, COS 1 or NIH 3T3 cells), or any of a number of plant hosts including, without limitation, algae, tree species, ornamental species, temperate fruit species, tropical fruit species, vegetable species, legume species, crucifer species, monocots, dicots, or in any plant of commercial or agricultural significance. Particular examples of suitable plant hosts include, but are not limited to, Conifers, Petunia, Tomato, Potato, Tobacco, Arabidopsis, Lettuce, Sunflower, Oilseed rape, Flax, Cotton, Sugarbeet, Celery, Soybean, Alfalfa, Medicago, Lotus, Vigna, Cucumber, Carrot, Eggplant, Cauliflower, Horseradish, Morning Glory, Poplar, Walnut, Apple, Grape, Asparagus, Rice, Maize, Millet, Onion, Barley, Orchard grass, Oat, Rye, and Wheat. In addition, as is discussed below, expression constructs may be expressed in a transgenic plant to turn on early pathogen response genes to enhance plant tolerance or resistance to pathogens and disease.

Materials for expressing these genes are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.). Descriptions and sources of useful host cells are also found in Vasil I. K., Cell Culture and Somatic Cell Genetics of Plants, Vol I, II, III Laboratory Procedures and Their Applications Academic Press, New York, 1984; Dixon, R. A., Plant Cell Culture—A Practical Approach, IRL Press, Oxford University, 1985; Green et al., Plant Tissue and Cell Culture, Academic Press, New York, 1987; and Gasser and Fraley, Science 244: 1293, 1989.

The method of transformation or transfection and the choice of vehicle for expression of the CDPK polypeptide or its protein kinase domain will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, K., Proc. Natl. Acad. Sci., U.S.A 87: 1228, 1990; Potrykus, I., Annu. Rev. Plant Physiol. Plant Mol. Biology 42: 205, 1991; and BioRad (Hercules, Calif.) Technical Bulletin #1687 (Biolistic Particle Delivery Systems). Expression vehicles may be chosen from those provided, e.g., in Cloning Vectors: A Laboratory Manual (P. H. Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

Most preferably, a CDPK polypeptide or its protein kinase domain is produced by a stably-transfected plant cell line, a transiently-transfected plant cell line, or by a transgenic plant. A number of vectors suitable for stable transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra). Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired nucleic acid sequence encoding a CDPK polypeptide or its protein kinase domain is obtained as described above, it may be manipulated in a variety of ways known in the art. For example, where the sequence involves non-coding flanking regions, the flanking regions may be subjected to mutagenesis.

The protein kinase domain sequence (or a CDPK polypeptide or protein kinase domain-containing fragment thereof), if desired, may be combined with other DNA sequences in a variety of ways. Such a sequence may be employed with all or part of the gene sequences normally associated with itself. In its component parts, a DNA sequence encoding a CDPK polypeptide or its protein kinase domain is combined in a DNA construct having a transcription initiation control region capable of promoting transcription and translation in a host cell.

In general, the constructs will involve regulatory regions functional in plants which provide for modified production of the regulator protein as discussed herein. The open reading frame coding for the regulator protein or functional fragment thereof will be joined at its 5′ end to a transcription initiation regulatory region such as the sequence naturally found in the 5′ upstream region of the CDPK polypeptide or its protein kinase domain. Numerous other transcription initiation regions are available which provide for constitutive or inducible regulation.

For applications where developmental, cell, tissue, hormonal, or environmental expression is desired, appropriate 5′ upstream non-coding regions are obtained from other genes, for example, from genes regulated during meristem development, seed development, embryo development, or leaf development.

Regulatory transcript termination regions may also be provided in DNA constructs of this invention as well. Transcript termination regions may be provided by the DNA sequence encoding the CDPK polypeptide or any convenient transcription termination region derived from a different gene source. The transcript termination region will contain preferably at least 1-3 kb of sequence 3′ to the structural gene from which the termination region is derived. Plant expression constructs having, for example, a CDPK protein kinase domain (e.g., the Arabidopsis CDPK2 protein kinase domain) as the DNA sequence of interest for expression may be employed with a wide variety of plant life. Such genetically-engineered plants are useful for a variety of industrial and agricultural applications as discussed herein. Importantly, this invention is applicable to dicotyledons and monocotyledons, and will be readily applicable to any new or improved transformation or regeneration method.

An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters. In most tissues of transgenic plants, the CaMV 35S promoter is a strong promoter (see, e.g., Odell et al., Nature 313: 810, 1985). The CaMV promoter is also highly active in monocots (see, e.g., Dekeyser et al., Plant Cell 2: 591, 1990; Terada and Shimamoto, Mol. Gen. Genet. 220: 389, 1990). Moreover, activity of this promoter can be further increased (i.e., between 2-10 fold) by duplication of the CaMV 35S promoter (see e.g., Kay et al., Science 236: 1299, 1987; Ow et al., Proc. Natl. Acad. Sci., U.S.A. 84: 4870, 1987; and Fang et al., Plant Cell 1: 141, 1989). In addition, the a minimal 35S promoter may also be used as is described herein.

Other useful plant promoters include, without limitation, the nopaline synthase promoter (An et al., Plant Physiol. 88: 547, 1988) and the octopine synthase promoter (Fromm et al., Plant Cell 1: 977, 1989).

For certain applications, it may be desirable to produce the CDPK polypeptide or its protein kinase domain in an appropriate tissue, at an appropriate level, or at an appropriate developmental time. For this purpose, there are an assortment of gene promoters, each with its own distinct characteristics embodied in its regulatory sequences, shown to be regulated in response to the environment, hormones, and/or developmental cues. These include gene promoters that are responsible for heat-regulated gene expression (see, e.g., Callis et al., Plant Physiol 88: 965, 1988; Takahashi and Komeda, Mol. Gen. Genet. 219: 365, 1989; and Takahashi et al., Plant J. 2: 751, 1992), light-regulated gene expression (e.g., the Arabidopisis Cab2 photosynthetic, leaf specific promoter described by Mitra at el., Plant Mol. Biol. 12: 169-179, 1989; the pea rbcS-3A described by Kuhlemeier et al., Plant Cell 1: 471, 1989; the maize rbcS promoter described by Schäffner and Sheen, Plant Cell 3: 997, 1991; or the cholorphyll a/b-binding protein gene found in pea described by Simpson et al., EMBO J. 4: 2723, 1985), hormone-regulated gene expression (for example, the abscisic acid (ABA) responsive sequences from the Em gene of wheat described by Marcotte et al., Plant Cell 1: 969, 1989; the ABA-inducible HVA1 and HVA22, and rd29A promoters described for barley and Arabidopsis by Straub et al., Plant Cell 6: 617, 1994, Shen et al., Plant Cell 7: 295, 1995; and wound-induced gene expression (for example, of wunI described by Siebertz et al., Plant Cell 1: 961, 1989), organ-specific gene expression (for example, of the tuber-specific storage protein gene described by Roshal et al., EMBO J. 6: 1155, 1987; the 23-kDa zein gene from maize described by Schernthaner et al., EMBO J. 7: 1249, 1988; or the French bean β-phaseolin gene described by Bustos et al., Plant Cell 1: 839, 1989; the vegetative storage protein promoter (soybean vspB) described by Sadka et al (Plant Cell 6: 737-749, 1994)), cycling promoters (e.g., the Arabidopsis cdc2a promoter described by Hemerly et al., Proc Natl Acad Sci USA 89: 3295-3299, 1992), senescence-specific promoters (e.g., the Arabidopsis SAG12 promoter described by Gan et al, Science: 270, 1986-1988, 1995), seed-specific promoters (for example, endosperm-specific or embryo-specific promoters), or pathogen-inducible promoters (for example, PR-1 or β-1,3 glucanase promoters).

Plant expression vectors may also optionally include RNA processing signals, e.g, introns, which have been shown to be important for efficient RNA synthesis and accumulation (Callis et al., Genes and Dev. 1: 1183, 1987). The location of the RNA splice sequences can dramatically influence the level of transgene expression in plants. In view of this fact, an intron may be positioned upstream or downstream of a CDPK polypeptide or its protein kinase-domain encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84: 744, 1987; An et al., Plant Cell 1: 115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Hoechst AG, Frankfurt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller, In: Genetic Engineering, vol 6, P W J Rigby, ed, London, Academic Press, 1987; and Lichtenstein, C. P., and Draper, J,. In: DNA Cloning, Vol II, D. M. Glover, ed, Oxford, IRI Press, 1985)), (2) the particle delivery system (see, e.g., Gordon-Kamm et al., Plant Cell 2: 603,1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23: 451, 1982; or e.g., Zhang and Wu, Theor. Appl. Genet. 76: 835, 1988), (5) liposome-mediated DNA uptake (see, e.g., Freeman et al., Plant Cell Physiol. 25: 1353, 1984), (6) electroporation protocols (see, e.g., Gelvin et al., supra; Dekeyser et al., supra; Fromm et al., Nature 319: 791, 1986; Sheen, Plant Cell 2: 1027, 1990; or Jang and Sheen, Plant Cell 6: 1665, 1994), and (7) the vortexing method (see, e.g., Kindle supra). The method of transformation is not critical to the invention. Any method which provides for efficient transformation may be employed. As newer methods are available to transform crops or other host cells, they may be directly applied.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, transfer and expression of transgenes in plant cells are now routine practices to those skilled in the art, and have become major tools to carry out gene expression studies in plants and to produce improved plant varieties of agricultural or commercial interest.

Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant; such techniques are described, e.g., in Vasil supra; Green et al., supra; Weissbach and Weissbach, supra; and Gelvin et al., supra.

In one particular example, a cloned protein kinase domain of a CDPK (or a CDPK polypeptide or a protein kinase-containing fragment thereof) construct under the control of the nos promoter and the nopaline synthase terminator and carrying a selectable marker (for example, kanamycin resistance) is transformed into Agrobacterium. Transformation of leaf discs (for example, of tobacco or potato leaf discs), with vector-containing Agrobacterium is carried out as described by Horsch et al. (Science 227: 1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al. supra; Gelvin et al. supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed and include PCR amplification assays using oligonucleotide primers designed to amplify only transgene RNA templates and solution hybridization assays using transgene-specific probes (see, e.g., Ausubel et al., supra). The RNA-positive plants are then analyzed for protein expression by Western immunoblot analysis using specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

In addition, if desired, once the recombinant CDPK polypeptide or its protein kinase domain is expressed in any cell or in a transgenic plant (for example, as described above), it may be isolated, e.g., using affinity chromatography. In one example, an anti-CDPK2 polypeptide antibody (e.g., produced as described in Ausubel et al., supra, or by any standard technique) may be attached to a column and used to isolate the polypeptide. Lysis and fractionation of CDPK2-producing cells prior to affinity chromatography may be performed by standard methods (see, e.g., Ausubel et al., supra). Once isolated, the recombinant protein can, if desired, be further purified, for example, by high performance liquid chromatography (see, e.g., Fisher, Laboratory Techniques In Biochemistry And Molecular Biology, eds., Work and Burdon, Elsevier, 1980).

Engineering Disease Resistant Plants

As discussed above, plasmid constructs designed for the expression of CDPK polypeptides are useful, for example, for activating plant defense pathways that confer anti-pathogenic properties to a transgenic plant. For example, CDPK genes that are isolated from a plant host (e.g., Arabidopsis, maize, or rice) may be engineered for expression in the same plant, a closely related species, or a distantly related plant species. For example, the cruciferous Arabidopsis CDPK2 gene may be engineered for constitutive low level expression and then transformed into an Arabidopsis host plant. Alternatively, the Arabidopsis CDPK2 gene may be engineered for expression in other cruciferous plants, such as the Brassicas (for example, broccoli, cabbage, and cauliflower). Similarly, a CDPK2 ortholog is useful for expression in any number of plants including grasses, brassicas, and solanaceous plants, such as tomato, potato, and pepper. To achieve pathogen resistance, it is important to express a CDPK protein at an effective level. Evaluation of the level of pathogen protection conferred to a plant by, for example, ectopic expression of a CDPK gene is determined according to conventional methods and assays.

In one working example, constitutive ectopic expression of the CDPK2 gene of Arabidopsis (Sheen, Science 274: 1900-1902, 1996) in Russet Burbank potato is used to control Phytophthora infestans infection. In one particular example, a plant expression vector is constructed that contains a CDPK2 cDNA sequence expressed under the control of the enhanced CaMV 35S promoter as described by McPherson and Kay (U.S. Pat. No. 5,359,142). This expression vector is then used to transform Russet Burbank potatp according to the methods described in Fischhoff et al. (U.S. Pat. No. 5,500,365). To assess resistance to fungal infection, transformed Russet Burbank and appropriate controls are grown to approximately eight-weeks-old, and leaves (for example, the second or third from the top of the plant) are inoculated with a mycelial suspension of P. infestans. Plugs of P. infestans mycelia are inoculated on each side of the leaf midvein. Plants are subsequently incubated in a growth chamber at 27° C. with constant fluorescent light.

Leaves of transformed Russet Burbank and control plants are then evaluated for resistance to P. infestans infection according to conventional experimental methods. For this evaluation, the number of lesions per leaf and percentage of leaf area infected are recorded every twenty-four hours for seven days after inoculation. From these data, levels of resistance to P. infestans are determined. Transformed potato plants that express a CDPK2 gene having an increased level of resistance to P. infestans relative to control plants are taken as being useful in the invention.

Alternatively, to assess resistance at the whole plant level, transformed and control plants are transplanted to potting soil containing an inoculum of P. infestans. Plants are then evaluated for symptoms of fungal infection (for example, wilting or decayed leaves) over a period of time lasting from several days to weeks. Again, transformed potato plants expressing the CDPK2 gene having an increased level of resistance to the fungal pathogen, P. infestans, relative to control plants are taken as being useful in the invention.

In another working example, expression of the Arabidopsis CDPK2 ortholog of tomato is used to control bacterial infection, for example, to Pseudomonas syringae. Specifically, a plant expression vector is constructed that contains the cDNA sequence of the CDPK2 ortholog from tomato is expressed under the control of the enhanced CaMV 35S promoter as described by McPherson and Kay, supra. This expression vector is then used to transform tomato plants according to the methods described in Fischhoff et al., supra. To assess resistance to bacterial infection, transformed tomato plants and appropriate controls are grown, and their leaves are inoculated with a suspension of P. syringae according to standard methods, for example, those described herein. Plants are subsequently incubated in a growth chamber, and the inoculated leaves are subsequently analyzed for signs of disease resistance according to standard methods. For example, the number of chlorotic lesions per leaf and percentage of leaf area infected are recorded and evaluated after inoculation. From a statistical analysis of these data, levels of resistance to P. syringae are determined. Transformed tomato plants that express a CDPK2 ortholog having an increased level of resistance to P. syringae relative to control plants are taken as being useful in the invention.

In still another working example, expression of the Arabidopsis CDPK2 ortholog from rice is used to control fungal diseases, for example, the infection of tissue by Magnaporthe grisea, the cause of rice blast. In one particular approach, a plant expression vector is constructed that contains the cDNA sequence of the rice CDPK2 ortholog, and is constitutively expressed under the control of the rice actin promoter described by Wu et al. (WO 91/09948). This expression vector is then used to transform rice plants according to conventional methods, for example, using the methods described in Hiei et al. (Plant Journal 6:271-282, 1994). To assess resistance to fungal infection, transformed rice plants and appropriate controls are grown, and their leaves are inoculated with a mycelial suspension of M. grisea according to standard methods. Plants are subsequently incubated in a growth chamber and the inoculated leaves are subsequently analyzed for disease resistance according to standard methods. For example, the number of lesions per leaf and percentage of leaf area infected are recorded and evaluated after inoculation. From a statistical analysis of these data, levels of resistance to M. grisea are determined. Transformed rice plants that express a rice CDPK2 otholog having an increased level of resistance to M. grisea relative to control plants are taken as being useful in the invention.

Non-Naturally Occurring Mutants

Plants having increased expression of a CDPK polypeptide are generated using standard mutagenesis methodologies. Such mutagenesis methods include, without limitation, treatment of seeds with EMS or fast neutron irradiation, as well as T-DNA insertion methodologies. Expression of a CDPK polypeptide and disease resistance phenotypes in mutated and non-mutated lines are evaluated according to standard procedures (for example, those described herein). When compared to non-mutated plants, mutated plants having increased expression of a gene encoding a CDPK polypeptide (for example, a CDPK2 ortholog) exhibit increased disease resistance relative to their non-mutated counterparts.

Conventional Plant Breeding

Once a non-naturally plant having an appropriate level of CDPK polypeptide expression and increased disease resistance has been obtained, conventional plant breeding methods can be used to transfer the CDPK structural gene and associated regulatory sequences via crossing and backcrossing. Such methods include crossing the disease resistant plant with a disease susceptible plant, recovering reproductive material from the progeny of the cross, and growing resistant plants from the reproductive material. If necessary, the agronomic characteristics of the susceptible plant is preserved by expanding this method to include the further steps of repetitively backcrossing the resistant progeny with susceptible plants, and selecting for expression of resistant phenotype among the progeny of the backcross, until the desired percentage of the characteristics of the susceptible plant are present in the progeny along with the gene imparting disease resistance.

Use

The invention described herein is useful for a variety of agricultural and commercial purposes including, but not limited to, improving a plant's resistance against plant pathogens, increasing crop yields, improving crop and ornamental quality, and reducing agricultural production costs. In particular, expression of a CDPK regulator polypeptide (for example, a constitutively-active CDPK2 or a protein kinase domain of a CDPK polypeptide) in a plant cell provides a method for activating early response pathogen genes to plant pathogens and can be used to protect plants from pathogen infestation that reduces plant productivity and viability.

The invention also provides for broad-spectrum pathogen resistance by facilitating the natural mechanism of host resistance. For example, CDPK can be expressed in plant cells at sufficiently high levels to activate a plant's defense response constitutively in the absence of signals from the acquired pathogen. The level of expression associated with such a plant defense response may be determined by measuring the levels of defense response gene expression as described herein or according to any conventional method. If desired, the transgenes are expressed by a controllable promoter such as a tissue-specific promoter, cell-type specific promoter, or by a promoter that is induced by an external signal or agent such as a pathogen- or wound-inducible control element, thus limiting the temporal or tissue expression or both of a systemic acquired resistance defense response. If desired, the genes designed to ectopically express a CDPK regulator polypeptide may also be expressed in roots, leaves, or fruits, or at a site of a plant that is susceptible to pathogen penetration and infection.

All publications and patent applications mentioned in this specification are herein incorporated by reference to the same extent as if each independent publication or patent application was specifically and individually indicated to be incorporated by reference.

What is claimed is:

1. A method of producing a plant having increased disease resistance, said method comprising the steps of:
a) providing a non-naturally occurring plant cell overexpressing a nucleic acid molecule encoding a calcium dependent protein kinase (CDPK) polypeptide; and
b) regenerating a plant from said plant cell, wherein said CDPK polypeptide is expressed in said plant, increasing the resistance of said plant to disease as compared to a naturally-occurring plant.

2. The method of claim 1, wherein said plant cell is a dicotyledonous plant cell.

3. The method of claim 2, wherein said dicotyledonous plant cell is a cruciferous plant cell.

4. The method of claim 1, wherein said plant cell is a monocotyledonous plant cell.

5. The method of claim 1, wherein said disease is caused by a plant pathogen.

6. The method of claim 1, wherein said non-naturally occurring plant cell is a transgenic plant cell.

7. The method of claim 6, wherein said transgenic plant cell comprises a trans gene that expresses a nucleic acid molecule encoding a CDPK polypeptide.

8. The method of claim 7, wherein said CDPK polypeptide is CDPK2.

9. The method of claim 7, wherein said CDPK polypeptide is CDPK4.

10. The method of claim 7, wherein said CDPK polypeptide consists essentially of a protein kinase domain.

11. The method of claim 7, wherein said CDPK polypeptide is a constitutively-active CDPK polypeptide.

12. The method of claim 7, wherein said transgene ectopically expresses said nucleic acid molecule encoding said CDPK polypeptide.

13. The method of claim 7, wherein the transgene comprises an inducible promoter.

14. The method of claim 7, wherein the transgene comprises a constitutive promoter.

15. The method of claim 7, wherein the transgene comprises a tissue-specific promoter.

16. The method of claim 7, wherein said nucleic acid molecule is either derived from Arabidopsis or is an ortholog thereof.

17. A method of conferring pathogen resistance on a plant, the method comprising the steps of:
a) crossing a pathogen resistant plant prepared by the method of claim 1 with a plant having susceptibility to a pathogen;
b) recovering reproductive material from the progeny of the cross; and
c) growing pathogen resistant plants from the reproductive material.

18. The method of claim 17, said method further comprising repetitively crossing the pathogen resistant progeny with disease susceptible plants, and selecting for expression of pathogen resistance.

19. A method for breeding pathogen resistance into plants, said method comprising:
a) selecting a plant that expresses a nucleic acid molecule encoding a CDPK polypeptide; and
b) selecting pathogen resistant progeny.

20. The method of claim 19, wherein said plant is a transgenic plant.

21. The method of claim 20, wherein said transgenic plant comprises a transgene that expresses a nucleic acid molecule encoding a CDPK polypeptide.

22. The method of claim 21, wherein said transgene ectopically expresses a nucleic acid molecule encoding said CDPK polypeptide.

23. The method of claim 21, wherein said CDPK polypeptide is CDPK2.

24. The method of claim 21, wherein said CDPK polypeptide is CDPK4.

25. The method of claim 21, wherein the CDPK polypeptide consists essentially of the protein kinase domain.

26. The method of claim 21, wherein the CDPK polypeptide is a constitutively-active CDPK polypeptide.

27. A non-naturally occurring plant that expresses a nucleic acid molecule encoding a CDPK2 polypeptide.

28. The non-naturally occurring plant of claim 27, said plant comprising a transgene that includes a nucleic acid molecule encoding a CDPK2 polypeptide, expression of said nucleic acid molecule being under the control of an expression control region that is functional in a plant cell.

29. The non-naturally occurring plant of claim 28, wherein the nucleic acid molecule encoding said CDPK2 polypeptide is derived from a plant.

30. The non-naturally occurring plant of claim 28, wherein the CDPK2 polypeptide consists essentially of the protein kinase domain.

31. The non-naturally occurring plant of claim 28, wherein said transgene that encodes said CDPK2 polypeptide is either derived from Arabidopsis or is an ortholog thereof.

32. The non-naturally occurring plant of claim 27, wherein said plant is a dicotyledonous plant

33. The non-naturally occurring plant of claim 27, wherein said plant is a monocotyledonous plant.

34. A seed from the non-naturally occurring plant of claim 27.

35. A cell from the non-naturally plant of claim 27.

36. A non-naturally occurring plant that expresses a nucleic acid molecule encoding a CDPK4 polypeptide.

37. The non-naturally occurring plant of claim 36, said plant comprising a transgene that includes a nucleic acid molecule encoding a CDPK4 polypeptide, expression of said nucleic acid molecule being under the control of an expression control region that is functional in a plant cell.

38. The non-naturally occurring plant of claim 36, wherein the nucleic acid molecule encoding said CDPK4 polypeptide is derived from a plant.

39. The non-naturally occurring plant of claim 36, wherein the CDPK4 polypeptide consists essentially of the protein kinase domain.

40. The non-naturally occurring plant of claim 36, wherein the CDPK4 polypeptide is a constitutively-active CDPK4 polypeptide.

41. The non-naturally occurring plant of claim 36, wherein said transgene that encodes said CDPK4 polypeptide is either derived from Arabidopsis or is an ortholog thereof.

42. The non-naturally occurring plant of claim 36, wherein said plant is a dicot.

43. The non-naturally occurring plant of claim 36, wherein said plant is a monocot.

44. A seed from the non-naturally occurring plant of claim 36.

45. A cell from the non-naturally occurring plant of claim 36.

46. A vector comprising an expression control region functional in plant cells operably linked to a nucleic acid molecule encoding a CDPK4 polypeptide.

47. A vector of claim 46 wherein the CDPK4 polypeptide consists essentially of the protein kinase domain.

48. The vector of claim 46 wherein the nucleic acid molecule encoding said CDPK4 polypeptide or protein kinase domain is derived from a plant.

49. The vector of claim 46, wherein nucleic acid molecule encoding said CDPK4 polypeptide is a constitutively-active CDPK4 polypeptide.

50. The vector of claim 46 wherein said nucleic acid molecule that encodes said CDPK4 polypeptide is either derived from Arabidopsis or is an ortholog thereof.

51. A cell comprising the vector of claim 46.

52. The cell of claim 51, wherein said cell is a plant cell.

53. The cell of claim 51, wherein said cell is a prokaryotic cell.


SEQUENCE LISTING (4 sequences)

SEQ ID NO: 1 - protein, 495 aa, Arabidopsis thaliana
METKPNPRRPSNTVLPYQTPRLRDHYLLGKKLGQGQFGTTYLCTEKSTSANYACKSIPKRKLVCREDYEDVWREIQIMHHLSEHPNVVRIKGTYEDSVFVHIVMEVCEGGELFDRIVSKGHFSEREAVKLIKTILGVVEACHSLGVMHRDLKPENFLFDSPKDDAKLKATDFGLSVFYKPGQYLYDVVGSPYYVAPEVLKKCYGPEIDVWSAGVILYILLSGVPPFWAETESGIFRQILQGKLDFKSDPWPTISEAAKDLIYKMLERSPKKRISAHEALCHPWIVDEQAAPDKPLDPAVLSRLKQFSQMNKIKKMALRVIAERLSEEEIGGLKELFKMIDTDNSGTITFEELKAGLKRVGSELMESEIKSLMDAADIDNSGTIDYGEFLAATLHMNKMEREEILVAAFSDFDKDGSGYITIDELQSACTEFGLCDTPLDDMIKEIDLDNDGKIDFSEFTAMMRKGDGVGRSRTMMKNLNFNIADAFGVDGEKSDD

SEQ ID NO: 2 - DNA, 1747 bp, Arabidopsis thaliana
gatccgggtacatattcttcttcttcttcaaatcgagatcgaagaagaaccaacaaaaaaccaaaaatggagacgaagccaaaccctagacgtccttcaaacacagttctaccatatcaaacaccacgattaagagatcattaccttctgggaaaaaagctaggccaaggccaatttggaacaacctatctctgcacagagaaatcaacctccgctaattacgcctgcaaatcgatcccgaagcgaaagctcgtgtgtcgcgaggattacgaagatgtatggcgtgagattcagatcatgcatcatctctctgagcatccaaatgttgttaggatcaaagggacttatgaagattcggtgtttgttcatattgttatggaggtttgtgaaggtggtgagctttttgatcggattgtttctaaaggtcattttagtgagcgtgaagctgtcaagcttattaagacgattcttggtgttgttgaggcttgtcattctcttggtgttatgcatagagatctcaaacctgagaatttcttgtttgatagtcctaaagatgatgctaagcttaaggctaccgattttggtttgtctgtcttctataagccaggacaatatttatatgacgtagttggaagtccgtactatgttgcaccagaggtgctaaagaaatgttatggacctgaaatagatgtgtggagtgctggtgttatcctctacattttactcagcggtgttcctcccttctgggcagagactgagtctggaatctttagacagatattgcaagggaagttagatttcaaatctgacccgtggcctactatctcagaagctgctaaagatttgatctataaaatgctcgaaaggagccccaagaaacgcatttctgctcatgaagccttgtgtcacccatggattgtcgatgaacaagcagcaccagacaagcctcttgatccagcagtcttatctcgtctaaagcagttttctcaaatgaataagattaagaaaatggcattacgggtaattgctgagagactttcagaggaagaaattggaggtctgaaggaattgttcaagatgatagacacagacaacagcggaacgattacttttgaagagctcaaagcgggtttgaagagagtcggatctgaactgatggaatcagaaatcaagtctctcatggatgcggctgatatcgacaacagtggtacaatagactacggagaattcctagcagcaaccttacacatgaacaagatggagagagaggagattctggtggctgcattttcggactttgacaaagacggaagcggttatatcaccatcgatgagcttcagtcagcttgcacagagtttggtctatgtgatacacctctggacgacatgatcaaggagattgatcttgacaatgacgggaagatcgatttctcggagtttacagcaatgatgaggaaaggagatggagttgggagaagcagaaccatgatgaagaacttgaacttcaacattgctgatgcttttggagttgatggtgaaaaatctgatgactgactcatcattcttccacaatttctgttttttttctctttaatttcgtttatattttgaattctaatttctaaggatacaaaaatatattctggcttgttttttgctttcctttttatttttgtacatgagcaactttctaaatttttatcctcatatggataatttttgcttcatataaaagtttttgaattcc

SEQ ID NO: 3 - protein, 501 aa, Arabidopsis thaliana
MEKPNPRRPSNSVLPYETPRLRDHYLLGKKLGQGQFGTTYLCTEKSSSANYACKSIPKRKLVCREDYEDVWREIQIMHHLSEHPNVVRIKGTYEDSVFVHIVMEVCEGGELFDRIVSKGCFSEREAAKLIKTILGVVEACHSLGVMHRDLKPENFLFDSPSDDAKLKATDFGLSVFYKPGQYLYDVVGSPYYVAPEVLKKCYGPEIDVWSAGVILYILLSGVPPFWAETESGIFRQILQGKIDFKSDPWPTISEGAKDLIYKMLDRSPKKRISAHEALCHPWIVDEHAAPDKPLDPAVLSRLKQFSQMNKIKKMALRVIAERLSEEEIGGLKELFKMIDTDNSGTITFEELKAGLKRVGSELMESEIKSLMDAADIDNSGTIDYGEFLAATLHINKMEREENLVVAFSYFDKDGSGYITIDELQQACTEFGLCDTPLDDMIKEIDLDNDGKIDFSEFTAMMKKGDGVGRSRTMRNNLNFNIAEAFGVEDTSSTAKSDDSPK

SEQ ID NO: 4 - DNA, 1657 bp, Arabidopsis thaliana
atggagaaaccaaaccctagaagaccctcaaacagtgttcttccatacgaaacaccaagattaagagatcactatctcctcggcaaaaagctaggccaaggccaatttggaacaacctatctctgtacagagaaatcatcatcagctaattacgcttgcaaatcaatcccaaaacgtaagcttgtatgtcgtgaagactacgaagatgtatggcgtgagattcagatcatgcatcatctctctgagcatcctaatgttgttagaatcaagggtacttatgaagactctgtttttgttcacattgttatggaagtttgtgaaggtggtgagctttttgatcggattgtttctaaagggtgttttagtgaacgtgaagctgctaagttgattaagactattcttggtgttgttgaggcttgtcattctcttggtgttatgcatagagatcttaagcctgagaatttcttgtttgatagtcccagtgatgatgctaagcttaaagctacagactttggtttgtctgtcttctacaagccagggcagtatctgtatgatgtagttggaagtccgtattatgttgcacctgaggttctgaagaaatgttatggaccagagatagacgtgtggagcgccggtgttatcttgtacatcttactaagtggggttcctcctttttgggcagaaaccgagtcaggaatctttaggcagatattgcaagggaagatagattttaaatctgatccgtggcctactatctcagaaggtgctaaagatttgatttacaaaatgctcgataggagccccaagaaacgtatttctgcacatgaagcattgtgtcacccttggattgttgatgaacatgctgcaccagacaagcctctcgacccagcagtcttgtcgcgacttaagcagttctcgcaaatgaataaaatcaagaaaatggccttacgagtaatcgcggagagactctcggaggaagagattggtggtctgaaggaattgttcaaaatgatagatacagacaacagtggaacaatcacctttgaagagcttaaagcaggtctaaagagagttggatctgaattgatggaatcagaaatcaagtctcttatggatgcggcggatatagacaacagtgggacaatagactacggtgaattcctagcagcgacattacatataaacaagatggagagagaagagaacttggtggttgcgttttcatactttgataaagatggtagcggttatatcaccattgacgagcttcaacaagcctgcacagagtttggtctctgtgacactcctcttgatgacatgatcaaagagattgatcttgataatgacgggaagattgatttctcagagtttactgctatgatgaagaaaggagatggtgttgggaggagcagaactatgaggaacaacttgaacttcaatatagctgaagcttttggagttgaggacacaagcagcactgctaaatctgatgattcaccaaagtaattataatcatctatatacttggaattgagaaatgagaactcacaaaagaaaaactgaatctttcctttttgttttcgtttccacttttgtagatgagcaactttctcaattttgttataagcatggataattttgcttcatattttctgcg